(19) United States
(12) Patent Application Publication
(10) Pub. No.: US 2022/0120759 A1
(43) Pub. Date: Apr. 21, 2022

(54) METHODS FOR DETECTION AND CHARACTERIZATION OF ANTI-VIRAL VECTOR ANTIBODIES

(71) Applicants: Rajiv MAHADEVAN, Redwood City, CA (US); Precision Bioservices, Inc., Redwood City, CA (US)

(72) Inventors: Eugen KOREN, Redwood City, CA (US); Tyler CHUANG, Redwood City, CA (US); Chad MORRIS, Redwood City, CA (US); M. Travis HARRISON, Redwood City, CA (US); Rajiv MAHADEVAN, Redwood City, CA (US)

(21) Appl. No.: 17/427,546

(22) PCT Filed: Jan. 31, 2020
(86) PCT No.: PCT/US2020/016053

§ 371 Date: Jul. 30, 2021

Related U.S. Application Data
(60) Provisional application No. 62/799,539, filed on Jan. 31, 2019.

(51) Int. Cl. G01N 33/68; G01N 21/552

(52) U.S. Cl. CPC G01N 33/6854; G01N 21/553

(57) ABSTRACT

Surface plasmon resonance-based methods for detecting and characterizing preexisting and/or treatment-induced anti-viral vector antibodies against viral vector-based gene therapy compositions in a biological sample from a subject are described.

### CROSS-REFERENCE TO RELATED APPLICATIONS

This patent claims priority to U.S. provisional application No. 62/799,539, filed Jan. 31, 2019, the disclosure of which is incorporated herein by reference in its entirety.

### FIELD OF THE INVENTION

This patent application relates to, among other things, non-cell-based methods of detecting antibodies against viral vector-based gene therapy compositions in biological samples.

### BACKGROUND OF THE INVENTION

Gene therapy is an increasingly important approach for addressing rare genetic diseases. According to the National Institutes of Health, about 7,000 rare diseases afflict up to 25 million Americans. An estimated 80% of these diseases are caused by a single-gene defect, and thus are potentially strong candidates for development of a target gene therapy to treat or cure the disease. However, developing safe and effective gene therapies for rare genetic disorders poses many challenges, including a paucity of subjects for undertaking clinical trials, and the existence of natural disease variations and sub-types among those subjects.

Gene therapy compositions generally utilize a vehicle or vector for delivering a gene-based therapy to a subject. Viral vectors, and particularly adeno-associated viral vectors (“AAVs”), are an increasingly widespread choice for the delivery of gene-based therapies. The use of different AAV serotypes, including wild type and variant AAV serotypes, is complicated, however, by the presence in individual subjects of preexisting antibodies, such as neutralizing antibodies, and/or the development in individual subjects of treatment-induced antibodies. These antibodies can neutralize, antagonize or inhibit viral transduction, and thus reduce the potential of AAV-based gene therapy drug responsiveness and efficacy. For an AAV-based therapeutic to reliably exert its maximal efficacy on a subject, it is necessary to screen and stratify patients in terms of their level of preexisting antibodies against AAV prior to administering the therapy, and/or to monitor development of treatment-induced antibodies after administering the therapy.

Current methods for detecting neutralizing antibodies in subjects include cell-based methods, whereby serum samples from a subject are used to challenge transduction of a cell culture by an AAV serotype test. These cell-based methods are time-consuming and cost- and materials-intensive, as they require on the order of 3 to 4 days of cell culturing, cell passage, cell washing, transduction, and incubation. These methods additionally utilize AAV vectors that include genetic material for molecular markers or fluorescent labels, or the like, to facilitate detection of transduced cells. Because such molecular markers or fluorescent labels are not intended in therapeutic compositions and are therefore not included in them, the test AAV vectors used in these cell-based methods differ from the optimized therapeutic AAV vectors that will ultimately be administered therapeutically.

Accordingly, novel methods of detecting preexisting and/or treatment-induced antibodies against viral vector-based gene therapy compositions are needed, including methods of detecting preexisting and/or treatment-induced antibodies against specific AAV serotypes in subjects. In addition, quantitative, high throughput methods of testing for antibodies against specific AAV serotypes in subjects are needed, as are methods for measuring concentration, association rates, and dissociation rates of antibodies for specific AAV serotypes in subjects.

### BRIEF SUMMARY OF THE INVENTION

It is an object of the present patent application to provide reliable and cost-efficient methods for analyzing biological samples from subjects and detecting preexisting as well as treatment-induced antibodies against viral vector-based gene therapy compositions, including preexisting neutralizing or total antibodies against specific AAV serotypes. Preferred methods include stratifying subject populations based on prevalence of preexisting anti-viral vector antibodies against viral vector-based gene therapy compositions and/or specific AAV serotypes in subjects. It is a further object of the present patent application to provide methods for measuring concentration and various characteristics of binding kinetics, including, but not limited to, association rates and dissociation rates of anti-viral vector antibodies for specific AAV serotypes in subjects.

Described herein are methods for detecting anti-viral vector antibodies, the methods comprising incubating a mixture that comprises a viral vector and a biological sample suspected of containing anti-viral vector antibodies against the viral vector; contacting the mixture with a ligand for the viral vector; and using surface plasmon resonance (SPR) sensor chip to determine the extent to which the viral vector associates with the ligand following the contacting step. In some embodiments, the association of the viral vector with the ligand is reduced in the presence of the anti-viral vector antibodies.

The methods described herein can further comprise using surface plasmon resonance to determine extents of association of the viral vector with the ligand in the presence of a negative control sample known to lack anti-viral vector antibodies against the viral vector, and comparing the association of the viral vector with the ligand in the presence of the biological sample suspected to contain anti-viral vector antibodies to that in the presence of the negative control sample.

In some embodiments, the anti-viral vector antibodies are neutralizing antibodies. In some embodiments, the anti-viral vector antibodies are immunoglobins (Igs) of class A (IgA), class D (IgD), class E (IgE), class G (IgG) or class M (IgM). In some embodiments, the anti-viral vector antibodies of class IgA are of isotype IgA1 or isotype IgA2 and/or the anti-viral vector antibodies of isotype IgG are of isotype IgG1, isotype IgG2, isotype IgG3 or isotype IgG4.

The methods described herein can further comprise contacting the incubated mixture with a complement system protein that is capable of binding to the anti-viral vector antibodies associated with the viral vector covalently immobilized on the SPR sensor chip. In some embodiments, the complement system protein is C1q. In some embodiments, the disclosed methods comprise determining the binding level of the complement system protein to the anti-viral vector antibodies, wherein the binding level is indicative of the complement system level of activation.

In some embodiments, the viral vector is an adeno-associated viral vector. In some embodiments, the viral vector is an AAV vector of serotype AAV1-AAV9, preferably of serotype AAV2, AAV5, AAV8, or AAV9. The viral vector can comprise a reporter such as a fluorescent or chemiluminescent reporter, for example, green fluorescent protein (GFP) and luciferase, respectively.

The methods described herein can comprise determining a rate of association of the viral vector to the ligand, determining a binding affinity of the viral vector to the ligand, and determining rates of association and dissociation of the viral vector and the ligand.

In some embodiments, the methods described herein comprise diluting the biological sample, determining the extent to which the viral vector associates with the ligand in the diluted biological sample, and comparing the extent of such association in the diluted biological sample. The methods can further comprise determining the concentration of the anti-viral vector antibody in the diluted biological sample and/or titering the anti-viral vector antibody in the biological sample.

The biological sample can be serum, plasma, whole blood, umbilical cord blood, cerebrospinal fluid, intraocular fluid, synovial fluid, saliva, bronchial fluid, alveolar fluid, gastrointestinal lavage fluids, and/or urine, for example.

In some embodiments, the ligand is immobilized on a solid support and the mixture flows over the solid support. The ligand can be covalently attached to the solid support. The solid support can comprise SPR chips coated with immobilized long and short chain dextran, carboxymethyl dextran, alginate, immobilized streptavidin, immobilized Protein A, immobilized nitrilotriacetic acid (NTA), or other coating suitable for use in SPR-based analysis. The ligand can be, for example, laminin receptor, heparan sulfate proteoglycans, AAV receptor, or an antibody specific to the viral vector. In some embodiments, the ligand is in native form. In some embodiments, the ligand is recombinant. The ligand can be biotinylated or poly-histidine tagged, or similarly modified with suitable affinity tags or markers.

The methods described herein can further comprise using surface plasmon resonance to determine association of the viral vector with the ligand in the presence of a positive control sample known to comprise anti-viral vector antibodies against the viral vector, and comparing the association of the viral vector with the ligand in the presence of the biological sample to the association of the viral vector with the ligand in the presence of the positive control sample.

In some embodiments, the positive control sample comprises a purified anti-viral vector antibody of known titer, of known affinity for the viral vector, and/or of known avidity for the viral vector. In some embodiments, comparing the association of the viral vector with the ligand in the presence of the biological sample to that in the presence of the positive control sample results in a quantitative measurement of the presence of anti-viral vector antibodies against the viral vector in the biological sample.

In some embodiments, using surface plasmon resonance to determine association of the viral vector with the ligand comprises assessing inhibition of the association by anti-viral vector antibodies in the biological sample or in the positive control sample based on Rmax values measured for a plurality of dilutions of the biological sample or the control sample.

Also described herein are methods comprising: obtaining a biological sample from each of multiple subjects suspected to be in need of treatment with a viral vector-based gene therapy composition; performing the methods for detecting anti-viral vector antibodies described herein on said samples; and stratifying the population of subjects based on the presence of anti-viral vector antibodies in the biological samples.

In some embodiments, subjects having biological samples determined to be negative for anti-viral vector antibodies or below a predetermined threshold for anti-viral vector antibodies are selected for treatment with the viral vector-based gene therapy composition. In some embodiments, subjects with tolerable levels of anti-viral vector antibodies are selected for treatment with the viral vector-based gene therapy composition.

In some embodiments, the subjects having biological samples determined to be positive for or above a predetermined threshold for anti-viral vector antibodies are excluded from treatment with the viral vector-based gene therapy composition. In one aspect, the methods described herein comprise providing a companion diagnostic for use with viral vector-based gene therapy compositions. Also described herein are kits for use as a companion diagnostic for a viral vector-based gene therapy composition.

### BRIEF DESCRIPTIONS OF THE DRAWINGS

The foregoing summary, as well as the following detailed description of preferred embodiments of the present patent application, will be better understood when read in conjunction with the appended drawings. It should be understood, however, that the application is not limited to the precise embodiments shown in the drawings.

FIGS. 1A-1B are series of diagrams depicting an example of surface plasmon resonance (SPR) assay according to the methods described herein. In FIG. 1A, an AAV-specific ligand 300 is immobilized to an SPR-suitable solid support 100 via covalent attachment 400. AAV vector particles 200 bind to the ligand to form a ligand-vector complex at the solid support surface. In FIG. 1B, AAV-specific neutralizing antibodies 500 competitively inhibit binding of the AAV particles to the ligand 300.

FIGS. 2A-2B are series of diagrams depicting an alternative example of surface plasmon resonance (SPR) assay according to the methods described herein. In FIG. 2A, an AAV-specific antibody 600 is immobilized to an SPR-suitable solid support 100 via covalent attachment 400. AAV vector particles 200 bind to the antibody 600 to form an antibody-vector complex at the solid support surface. In FIG. 2B, AAV-specific neutralizing antibodies 500 competitively inhibit binding of the AAV particles to the antibody 600.

FIG. 3 is a graph depicting an example sensogram output of a competitive inhibition surface plasmon resonance assay where the Rmax values are inversely proportional to the levels of inhibition according to the methods described herein.

FIG. 4 is a series of a graph and a table depicting the binding of anti-AAV8-specific neutralizing mouse monoclonal IgG2a antibody (ADK8) and the confirmatory, isotype specific anti-mouse IgG2a antibodies to the AAV8 virus sensor chip. Three separate sensogram cycles were performed and displayed four different events (A, B, C, D) in a practically overlapping pattern. Section A showed virtually no binding of the unspiked negative serum pool to the virus surface representing the sensor chip background. Section B showed significant binding of ADK8 relative to baseline with the mean RU of 606.26±5.45 SD and 0.89% CV. Section C showed binding of anti-IgG2a confirmatory antibody with the mean RU of 922.3±11.88 SD and 1.28% CV relative to baseline, which included additive ADK8 and confirmatory anti-IgG2a binding. Section D demonstrated regeneration of the virus sensor chip.

FIG. 5 is a graph depicting anti-Mouse IgG2a response in serum samples with and without ADK8. Four sensograms were generated: (1) NAb negative serum spiked with ADK8 (168.7 RU); (2) NAb positive serum spiked with ADK8 (149.9 RU); (3) NAb negative serum unspiked with ADK8 (6.5 RU); and (4) NAb positive unspiked with ADK8 (6.0 RU).

FIG. 6 is a graph depicting the reactivity of anti-human IgA/G/M cocktail with human serum antibodies bound to the immobilized AAV8 virus chip. Serum samples tested include neutralizing antibody anti-AAV (NAb) negative serum pool (NC Pool) and 5 previously identified NAb-positive sera. Sensograms (numbered 1 to 6) represent different levels of the anti-human IgA/G/M response to serum antibodies bound to the AAV8 virus in ascending order from lowest to the highest: NC Pool (#6); BRH1454926 (#5); BRH1454987 (#1); BRH1454904 (#3); BRH1454903 (#2) and BRH145910 (#4).

FIG. 7 is a graph depicting the reactivity of human C1q with human serum antibodies bound to the immobilized AAV8 virus chip. Serum samples tested include NAb negative serum pool (NC Pool) and 5 previously identified NAb-positive sera (BRHs). Sensograms (numbered 1 to 6) represent different levels of the C1q response to serum antibodies bound to the AAV8 virus in ascending order from lowest (51 RU) to the highest (136 RU): BRH1454903 (#2); BRH145910 (#4); BRH1454926 (#5); BRH1454904 (#3); BRH1454987 (#1) and NC Pool (#6).

### DETAILED DESCRIPTION OF THE PREFERRED EMBODIMENTS

Discussion of protocols, documents, acts, materials, devices, articles or the like included in the present patent application is for the purpose of providing context for the described subject matter. Such discussion is not an admission that any or all of these matters form part of the prior art with respect to any subject matter disclosed or claimed.

Unless defined otherwise, all technical and scientific terms used herein have the same meaning as commonly understood by those skilled in the art to which this patent application pertains. Otherwise, certain terms used herein have the meanings as set forth herein.

As used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes f 10% of the recited value. For example, a concentration of 1 mg/mL includes 0.9 mg/mL to 1.1 mg/mL. Likewise, a concentration range of 1% to 10% (w/v) includes 0.9/6 (w/v) to 11% (w/v). As used herein, the use of a numerical range expressly includes all possible subranges, all individual numerical values within that range, including integers within such ranges and fractions of the values unless the context clearly indicates otherwise. Unless otherwise indicated, the term “at least” preceding a series of elements is to be understood to refer to every element in the series.

The terms “about,” “approximately,” “generally,” “substantially,” and like terms, when used herein referring to a dimension or characteristic of a component of the disclosed subject matter, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Those skilled in the art will recognize or be able to ascertain using no more than routine experimentation, many equivalents to the specific embodiments of the subject matter described herein. Such equivalents are intended to be encompassed by this patent application.

A composition, a mixture, a process, a method, an article, or an apparatus described herein that is said to comprise a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning and therefore satisfy the requirement of the term “and/or.”

The term “antibody,” “total antibody,” and like terms is meant in a broad sense and includes immunoglobulin molecules including, monoclonal antibodies, antibody fragments, bispecific or multispecific antibodies, dimeric, tetrameric or multimeric antibodies, and single chain antibodies. Immunoglobulins can be assigned to five major classes, namely IgA, IgD, IgE, IgG, and IgM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (κ) and lambda (λ), based on the amino acid sequences of their constant domains.

As used herein, “biological sample” refers to a fluid sample or a tissue sample from a subject. Examples of biological samples include is serum, plasma, whole blood, umbilical cord blood, cerebrospinal fluid, intraocular fluid, synovial fluid, saliva, bronchial fluid, alveolar fluid, gastrointestinal lavage fluids, and/or urine, for example.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

A used herein, “complement system” refers to distinct plasma proteins (more than 30) that interact with one another and enhance the ability of antibodies and phagocytic cells to clear pathogens. A classical complement pathway typically requires antigen-antibody complexes for activation. An alternative pathway can be activated by spontaneous complement component 3 (C3) hydrolysis, foreign material, pathogens, or damaged cells

As used herein, “C1q” or “Complement C1q” is the first subcomponent of the C1 complex of the classical pathway of the complement system activation.

As used herein, “gene therapy composition” refers to any therapeutic agent designed to treat or alleviate symptoms of a genetic disease. Gene therapy compositions can include viral vectors, including adenovirus associated vectors (AAV), and variants and serotypes thereof, for example AAV1, AAV2, AAV3, AAV4, AAV5, AAV6, AAV7, AAV8, and or AAV9, preferably AAV2, AAV5, AAV8, and/or AAV9.

In some embodiments of the present methods, the gene therapy compositions are for use in clinical studies to determine the safety and efficacy of the gene therapy composition in a subject population. In some embodiments of the present methods, the gene therapy compositions are for use in non-clinical studies to determine the safety and efficacy of the gene therapy composition in animals.

As used herein, “neutralizing antibody” or “NAb” refers to antibodies against a particular gene therapy composition, including antibodies against viral vectors used in gene therapy compositions. Neutralizing antibodies can be present in a subject or a biological sample from a subject. Neutralizing antibodies can be specific to a particular variant or serotype of a gene therapy composition or viral vector. For example, neutralizing antibodies can be preexisting antibodies and/or treatment-induced antibodies against a particular serotype of an adeno-associated viral vector. The neutralizing antibodies in a subject or a biological sample from a subject inhibit or neutralize the gene therapy composition that is administered to the subject or to cells otherwise susceptible to viral transduction. In this manner, neutralizing antibodies against particular gene therapy compositions can reduce the efficacy or increase the risk of administering the particular gene therapy composition to a subject.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, Rhesus monkeys, Cynomolgus monkeys, humans, etc., preferably a human. The term “subject” may refer to any subject suspected to be in need of treatment with a viral vector-based gene therapy composition for treatment or alleviation of any condition amenable to gene-based therapeutic intervention. “Multiple subjects” or a “population of subjects” can refer to any group of subjects considered for inclusion in a clinical study of a viral vector-based gene therapy composition.

As used herein, the term “surface plasmon resonance” or “SPR” refers to the physical phenomenon and also to the detection method whereby incident light directed at a target conducting surface stimulates a plasmon wave of oscillating electrons at the conducting surface, which oscillation is sensitive to molecular changes at the surface, including adsorption of molecules at the surface. Suitable instruments, surfaces, and materials for conducting SPR are known. For example, preferred systems for conducting SPR include Biacore SPR systems, Horiba Scientific SPR imaging systems, BioNavis MP-SPR Navi, Bruker Sierra SPR-32, ForteBio Pioneer SPR, and BioRad Proteon SPR systems.

“Stratifying” a population of subjects, as used herein, refers to qualitatively and/or quantitatively assessing one or more physical or genetic characteristics of each member of the population to assess or to predict each member's responsiveness to a gene therapy composition. For example, each member of a population of subjects considered for treatment with or inclusion in a clinical study of a particular gene therapy composition can be assessed for the presence or absence of anti-viral vector antibodies against the gene therapy composition, and the population in this manner is stratified into classes based on predicted responsiveness to the gene therapy composition. In this example, classes assessed to have high levels of anti-viral vector antibodies against the gene therapy composition preferably would not be administered the composition, whereas classes assessed to have no or low levels of anti-viral vector antibodies would be selected for administration of the composition.

Stratifying a population of subjects can be based on quantitative and/or qualitative assessments of one or more physical or genetic characteristics. Qualitative assessments can include, for example, age, sex, health status, disease history, or other relevant considerations of the subject population. Quantitative assessments can include, for example, presence in the subject population of particular neutralizing antibodies, including concentration, affinity, avidity, and other relevant characteristics of antibodies against a particular gene therapy composition. For instance, members of a subject population can be assessed for the presence or absence of neutralizing antibodies against a particular gene therapy composition, and, if present, the neutralizing antibodies can be quantitatively characterized in terms of affinity, avidity, or other relevant characteristics. These quantitative measures of the anti-viral vector antibodies can be used to establish pre-determined thresholds for stratifying subject populations. Members of the population negative for anti-viral vector antibodies or having neutralizing antibodies below the pre-determined threshold can be favorably considered for treatment with the gene therapy composition, whereas members positive for neutralizing antibodies or having neutralizing antibodies above the pre-determined threshold are preferably withdrawn from consideration for treatment with the gene therapy composition.

Relevant characteristics of antibodies, as referred to herein, include, but are not limited to, presence or absence of an antibody specific for a particular antigen (i.e., viral vector), specificity for a particular antigen (i.e., viral vector), binding kinetics, affinity, and concentration. For example, relevant characteristics include rate of association (kₒₙ), rate of dissociation (k_{off}), and binding constant/binding affinity (or “equilibrium dissociation constant,” K_{D}) (equal to k_{off}/kₒₙ) as well as concentration of a given antibody in a biological sample. The same parameters (kₒₙ; k_{off}; k_{off}/kₒₙ; concentration) allow for detailed characterization of the virus-ligand binding in the presence and/or absence of anti-viral vector anti-virus antibody.

Described herein are surface plasmon resonance-based anti-viral vector antibody (NAb) or total antibody (TAb) assays for viral vector-based gene therapies. The viral vector-based gene therapies can be adeno-associated virus (AAV) therapeutics. The described methods and assays can be used to detect and measure the levels of preexisting and/or treatment-induced NAbs against a particular viral vector serotype in a subject. For example, the described methods and assays can be used to detect preexisting anti-viral vector antibodies against AAV serotypes, for example AAV2, AAV5, AAV8, and/or AAV9 in a subject. In addition, the described methods and assays can be used to characterize the binding characteristics of these viruses to their respective ligands (i.e., immobilized receptors or antibodies) in the presence and/or absence of neutralizing antibodies. In this manner, the described methods can facilitate the selection of subjects for treatment with and/or for inclusion in clinical trials of particular gene therapy compositions. The described methods can also facilitate determination of effective dose or optimization of dosing regimen of gene therapy compositions for individual subjects based on the presence and characteristics of particular anti-viral vector antibodies (NAbs and/or TAbs) in the subjects.

The described assays and methods utilize surface plasmon resonance (SPR) to detect anti-viral vector antibodies in a sample, and/or to characterize effects of anti-viral vector antibodies in a sample on binding kinetics between the virus and its receptor. These assays and methods can be used to screen and stratify subjects in terms of their level of preexisting anti-viral vector antibodies to specific AAV serotypes, for example. In some embodiments, the assays and methods can be used to assess and monitor levels of treatment-induced anti-viral vector antibodies to specific AAV serotypes. In some embodiments, the assays described herein can be a companion diagnostic for use with a particular viral vector-based gene therapy composition. For example, the companion diagnostics can be used to detect antibodies against particular AAV-based gene therapy compositions, and thereby to screen and stratify subjects for treatment and/or for inclusion in clinical studies to test AAV-based gene therapy compositions.

The described assays and methods for characterizing anti-viral vector antibodies via SPR are preferably carried out with viral vectors intended for therapeutic administration. In some embodiments, the characterization of anti-viral vector antibodies (e.g. anti-AAV antibodies) can be achieved with wild type viral vectors (e.g. wild type AAVs) of corresponding serotypes.

The described methods for detecting anti-viral vector antibodies can comprise incubating a mixture that comprises a viral vector and a biological sample suspected of containing anti-viral vector antibodies against the viral vector. In some embodiments, the viral vector is an adeno-associated viral vector (AAV), and in particular, AAV-based gene therapy compositions. Example AAV-based gene therapy compositions include, but are not limited to, AAV2-, AAV5-, AAV8-, and AAV9-based gene therapy compositions.

The mixture comprising the viral vector and the biological sample can be contacted with a ligand for the viral vector. In some embodiments, contacting the mixture with the ligand can comprise, for example, flowing the mixture over a surface comprising the ligand. In some embodiments, the ligand is immobilized on a solid support and the mixture flows over the solid support. The ligand can be immobilized via covalent attachment to the solid support. In some embodiments, the ligand can be immobilized to the solid support via immunogenic affinity to the solid support or to a secondary ligand bound to the solid support. Suitable materials for the solid support include plastic, glass, or metallic substrates having a gold film deposited on a surface thereof. The gold film can be coated with, for example, long and short chain dextran, carboxymethyl dextran, alginate, immobilized streptavidin, immobilized Protein A, immobilized NTA (nitrilotriacetic acid), or other suitable coating. The ligand can be immobilized to, for example, the long chain carboxymethyl dextran or other suitable material on the solid support.

Suitable methods for immobilizing the ligand on the solid support will depend in part on the materials and coatings selected for the solid support, but include, without limitation, amine coupling; carbodiimide coupling; glutaryl aldehyde cross-linking; sulfhydryl cross-linking; cyanogen bromide activated coupling; chelation coupling; biotin-avidin/biotin-streptavidin binding; immunoglobulin-protein A/G/L binding; or polyhistidine-NTA binding.

In the methods described herein, the ligand can be any suitable ligand having binding specificity to the viral vector, and the ligand will vary depending on the viral vector selected for use in the described methods. In some embodiments, the ligand is a cellular receptor for the viral vector. Cellular receptors for viral vectors include, but are not limited to, naturally occurring cell surface proteins, carbohydrates, proteoglycans, or other molecules, or synthetic fragments, hybrids, or conjugates thereof to which viral vectors specifically bind. Under normal cellular conditions, binding of a viral vector to such a receptor can enable viral infection of the cell. In some embodiments, the ligand is an antibody or fragment thereof that specifically binds to the viral vector. Example ligands include, but are not limited to, the laminin receptor, heparan sulfate proteoglycans, AAV receptor, or an antibody specific to the viral vector.

In the described methods, contacting the mixture comprising the viral vector and the biological sample with the ligand will result in association of the viral vector with the ligand. The association of the viral vector with the ligand can be immunospecific or other specific binding. Said association can be detected using surface plasmon resonance. The extent to which the viral vector associates with the ligand can also be determined using surface plasmon resonance. In the event that anti-viral vector antibodies (e.g. neutralizing antibodies or total antibodies) specific for the viral vector are present in a biological sample, said anti-viral vector antibodies will compete with the ligand for association with the viral vector. In some embodiments, the presence of anti-viral vector antibodies will inhibit or reduce association of the viral vector with the ligand. Anti-viral vector antibodies in the biological sample can competitively inhibit association of the viral vector with the ligand. Thus, in the presence of anti-viral vector antibodies in the biological sample, the association of the viral vector with the ligand will be reduced, and detection of the association will be reduced.

In some embodiments of the methods described herein, surface plasmon resonance can be used to determine association of the viral vector with the ligand in the presence of a negative control sample. The negative control sample can be pooled from a plurality of biological samples from one or more subjects known to be negative for anti-viral vector antibodies. The negative control sample can be a synthetic sample known to be negative for anti-viral vector antibodies against the viral vector. Use of a negative control sample in this manner would not be expected to reduce association of the viral vector with the ligand via competitive inhibition. Comparing the association of the viral vector with the ligand in the presence of the biological sample to that in the presence of the negative control sample can thus facilitate determining whether the biological sample contains anti-viral vector antibodies.

The viral vector utilized in the methods described herein can comprise a reporter or a gene encoding a reporter. The reporter gene can be operably linked to a promoter region for driving expression of the reporter. In some embodiments, the promoter region is a human-derived gene expression promoter. The reporter gene can be any suitable reporter, including, for example, known fluorescent or chemiluminescent reporters, affinity tags, colorimetric reporters, or antigens. The reporter can be, for example, a fluorescent or chemiluminescent reporter. In some embodiments, the reporter is luciferase. Use of a viral vector having a reporter can facilitate comparison of the viral vector's response in the methods described herein compared to the viral vector's response in alternative, for example, cell-based assays for detecting anti-viral vector antibodies. In this way, use of viral vectors having reporters in the disclosed non-cell-based assays can be validated against or otherwise compared to alternative, for example, cell-based assays. Use of viral vectors having reporters will have additional benefits, including, for example, post-assay detection, isolation, and visualization of the viral vectors. Reporters, however, are not needed to perform the methods described herein. In some cases, it will be advantageous to use viral vectors lacking reporters, as these viral vectors will better represent, or in some cases can be identical to, viral vectors employed in clinical settings for use as gene therapy compositions; whereas, viral vectors comprising reporters are usually considered a proxy for the clinical version of the viral vector, which should generally lack the reporter.

Surface plasmon resonance can be used as described herein to characterize the binding kinetics of the viral vector and the anti-viral vector antibody. For example, SPR can be used in the described methods to determine a rate of association (kₒₙ) of the viral vector to the ligand. SPR can be used to determine a rate of dissociation (k_{off}) of the viral vector from the ligand. SPR can be used to determine a binding affinity of the viral vector to the ligand (or “equilibrium dissociation constant,” K_{D}) (equal to k_{off}/kₒₙ) in the presence and/or absence of anti-viral vector antibodies. SPR assays can be repeatedly run using the same biosensor chip after an appropriate washing-off and regeneration of the immobilized ligand. SPR can be used to determine the concentration of anti-viral vector antibodies in a biological sample, or to determine the titer of anti-viral vector antibodies in a biological sample, or to determine the titer of viral vector required to achieve an efficacious dose of the viral vector in a subject.

Diluting the biological sample and performing the described methods on increasing dilutions of the biological sample will permit more accurate characterization of the biological sample and any anti-viral vector antibodies therein. In some embodiments, the methods comprise preparing serial dilutions of the biological sample and performing the described methods on the serial dilutions. Comparing the extent of association of the viral vector with the ligand will be facilitated by determining and comparing the extent of competitive inhibition of dilutions or serial dilutions of the biological sample.

The methods described herein can comprise using surface plasmon resonance to determine association of a viral vector with a ligand in the presence of a positive control sample. The positive control sample, for example, can be a biological or other sample known to comprise anti-viral vector antibodies against the viral vector. Comparing association of the viral vector with the ligand in the presence of the biological sample to that in the presence of the positive control sample will facilitate determination of the presence of and the concentration of anti-viral vector antibodies in the biological sample. For instance, in some embodiments, the positive control sample comprises a purified anti-viral vector antibody of known titer. In some embodiments, the purified anti-viral vector antibody is of known affinity for the viral vector, and/or the purified anti-viral vector antibody is of known avidity for the viral vector. Comparing the association of the viral vector with the ligand in the presence of the biological sample to that in the presence of the positive control sample can thus result in a quantitative measurement of the presence of anti-viral vector antibodies against the viral vector in the biological sample. Similarly, the use of a positive control sample in this manner can result in quantitative measurement of the binding kinetics of the vector in the presence of anti-viral vector antibodies in a biological sample.

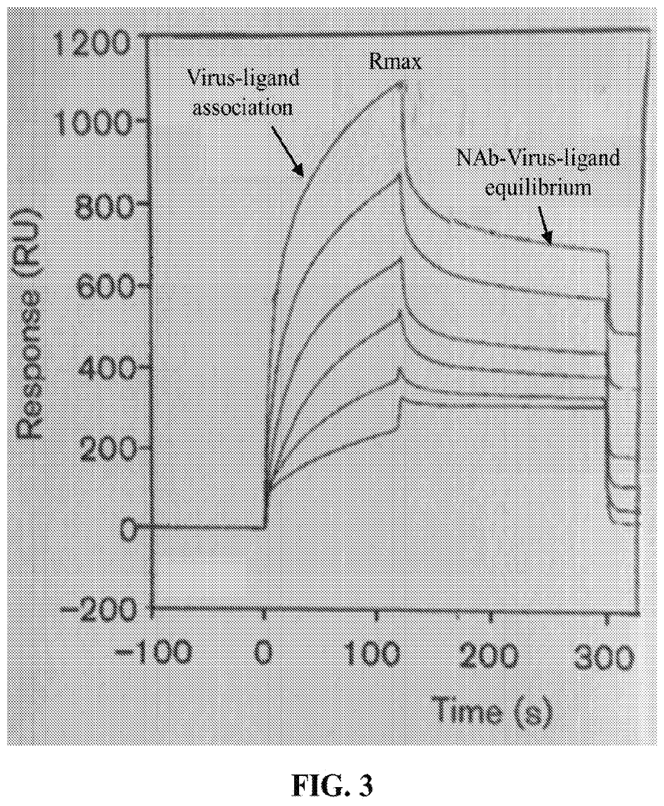

The use of surface plasmon resonance and competitive inhibition assays using surface plasmon resonance as described herein can result in detection and characterization of binding events involving the viral vectors, ligands specific for the viral vectors, and anti-viral vector antibodies specific for the viral vectors. For example, using surface plasmon resonance to determine association of a viral vector with a ligand in accordance with the methods described herein can include assessing inhibition of the association by neutralizing antibodies in the biological sample, and/or assessing inhibition of the association by neutralizing antibodies in the positive control sample. An example output of a surface plasmon resonance assay is a sensogram as depicted in FIG. 3. A typical sensogram depicts a curve with different aspects of the curve representing different binding events. In the methods described herein, a typical sensogram will depict an initial steady state SPR output (measured in relative response units) prior to contacting the ligand with the viral vector, followed by an association curve depicting the change in SPR output as a function of ligand-viral vector association upon contact by the viral vector, followed by maximal binding of the viral vector (“Rmax”), followed by spontaneous dissociation of the viral vector-ligand complex. A typical sensogram also depicts regeneration of the immobilized ligand after an appropriate washing-off of the viral vector.

In some embodiments, regeneration of a ligand immobilized on the surface of the solid support (e.g., sensor chip) is achieved by running an appropriate reagent, e.g., a washing reagent, over the solid support after the binding of the vector to the ligand is completed and acceptable sensogram obtained. Suitable washing reagents include: low pH reagents (e.g., 10 mM glycine-HCl at pH 1.5 to 3), high pH reagents (e.g., 1 to 100 mM NaOH at pH 10.98 to 12.88), high ionic strength reagents (e.g., up to 5 M NaCl or 4 M MgCl₂), low concentration detergents (e.g., SDS up to 0.5%), or ethylene glycol at concentrations up to 100%.

The vector binding step and subsequent regeneration make a complete cycle, after which the same chip can be used for another binding event. Depending on the stability of the ligand's attachments to the solid support surface and regeneration method, the same chip can be used repeatedly (e.g., up to 100 cycles in some cases) to generate reproducible sensograms.

Thus, SPR output in the form of sensograms can be used to assess multiple characteristics of anti-viral vector antibodies in a biological sample (such as neutralizing antibodies). Detailed assessment by means of surface plasmon resonance can be achieved, for example, by measuring Rmax values for a plurality of dilutions of the biological sample or the control sample. “Rmax value,” as used herein, refers to the maximum relative response measured for association of the viral vector with the ligand at a given concentration or dilution of biological sample/positive control sample, as measured in arbitrary response units using the methods as described herein.

In some embodiments, performing the SPR assays described in this application involves selecting an appropriate ligand such as a virus receptor or an anti-virus antibody, and purifying and tagging it, if desired. The assays can further involve selecting an appropriate sensor chip, determining the pH and ligand concentration as well as selecting the coupling method for optimal ligand immobilization on the surface of the sensor chip. After such surface preparation, optimal virus concentration can be determined and, in some embodiments, flowed across the chip in absence of anti-viral vector antibody in order to generate a sensogram displaying the maximal virus binding (Rmax). Rmax can represent 100% binding of the virus on a chip as described/optimized, and can be used to normalize assessment of relative virus binding in the presence of antibodies. To determine the level of anti-viral vector anti-virus antibodies, virus particles can be pre-incubated with serial dilutions of unknown serum samples. Such pre-incubations can be carried out with known concentrations of an anti-viral vector antibody positive control expressed in mass units. Both pre-incubated serum and positive control samples can be run over the immobilized ligand to generate respective sensograms. For each positive control antibody concentration and serum dilution, corresponding Rmax values can be recorded. Rmax values decreasing in proportion with the levels of anti-viral vector antibodies reflect inhibitory effects of antibodies on the virus-ligand binding. Using Rmax values versus corresponding serum dilutions, the level of anti-virus antibody levels can be assessed and expressed in titer units. Alternatively, Rmax values recorded against the corresponding known concentrations of the positive control antibody can allow for the construction of a standard curve, which can be used to determine actual concentration of anti-viral vector antibodies in serum samples expressed in mass units. In addition, from the sensogram components, affinity, avidity, and kinetics of the virus-ligand or virus-antibody binding can be analyzed using appropriate algorithms provided, for example, by the instrument manufacturers.

The present patent application further relates to methods of selecting for treatment one or more subjects from a population of subjects suspected of being in need of treatment with a viral vector-based gene therapy composition. In some embodiments, the methods comprise obtaining a biological sample from each of multiple subjects in the population of subjects suspected to be in need of treatment with the viral vector-based gene therapy composition; performing on said samples the methods described herein to detect and/or characterize anti-viral vector antibodies; and stratifying the population of subjects based on the presence of anti-viral vector antibodies in the biological samples. Subjects having biological samples determined in this manner to be negative for anti-viral vector antibodies or below a predetermined threshold for anti-viral vector antibodies are selected for treatment with the viral vector-based gene therapy composition, whereas subjects having biological samples determined to be positive for or above a predetermined threshold for anti-viral vector antibodies are excluded from treatment.

In one aspect, the methods described herein are performed as a companion diagnostic for use with the viral vector-based gene therapy composition. The companion diagnostic can be used to screen for and exclude from clinical studies or treatment subjects having preexisting anti-viral vector antibodies against the viral vector-based gene therapy composition. Alternatively, or additionally, the companion diagnostic can be used to screen for and exclude from clinical studies or treatment subjects who were initially negative for the preexisting anti-viral vector antibodies against the viral vector-based gene therapy composition but developed such antibodies in response to the administration of a given vector. In this way, the described methods can be used to stratify a population of subjects to ensure that only subjects who are negative for preexisting anti-viral vector antibodies against a viral vector-based gene therapy composition are included in clinical studies of and/or for treatment with the gene therapy composition. In some situations, subjects with low levels of preexisting anti-viral vector antibodies may be included in clinical studies. Thus, the methods described herein can improve the reliability of clinical studies and the therapeutic outcome of treatment with viral vector-based gene therapy compositions.

Also disclosed herein are kits or articles of manufacture. The kits or articles of manufacture can include, for example, reagents for performing the methods described herein, including viral vectors, negative control samples, positive control samples, solid supports, buffers, diluents, etc. The kits can also include instructions for using the reagents in the methods described herein to detect anti-viral vector antibodies in a biological sample. The kits can also include a package or container that is compartmentalized to receive one or more reagents or articles for use in the methods described herein. The articles of manufacture provided herein can contain packaging materials. A kit typically also includes labels listing contents and/or instructions for use, and package inserts with instructions for use. A set of instructions will also typically be included. Thus, the articles of manufacture provided herein can further contain a package insert that contains instructions or assay protocol steps for performing the methods described herein. In one aspect, the kits are for use as a companion diagnostic for a viral vector-based gene therapy composition.

Those skilled in the art will appreciate that numerous changes and modifications can be made to the preferred embodiments of the invention and that such changes and modifications can be made without departing from the spirit of the invention. It is intended that the appended claims cover all such equivalent variations fall within the true spirit and scope of the invention.

### ILLUSTRATIVE EMBODIMENTS

Provided here are illustrative embodiments of the disclosed technology. These embodiments are illustrative only and do not limit the scope of the present disclosure or of the claims attached hereto.

Embodiment 1. A method for detecting an anti-viral vector antibody, the method comprising:
incubating a mixture that comprises a viral vector and a biological sample suspected of containing an anti-viral vector antibody against the viral vector;
contacting the mixture with a ligand for the viral vector; and
using surface plasmon resonance (SPR) sensor chip to determine the extent to which the viral vector associates with the ligand following said contacting step;
wherein the association of the viral vector with the ligand is reduced in the presence of the anti-viral vector antibody.

Embodiment 2. The method of embodiment 1, further comprising using SPR to determine association of the viral vector with the ligand in the presence of a negative control sample known to lack an anti-viral vector antibody against the viral vector, and comparing the association of the viral vector with the ligand in the presence of the biological sample to that in the presence of the negative control sample.

Embodiment 3. The method of embodiment 1 or 2, wherein the anti-viral vector antibody is a neutralizing antibody.

Embodiment 4. The method of embodiment 1 or 2, wherein the anti-viral vector antibody is an immunoglobin (Ig) of class A (IgA), class D (IgD), class E (IgE), class G (IgG) or class M (IgM).

Embodiment 5. The method of embodiment 4, wherein the anti-viral vector antibody of class IgA is of isotype IgA1 or isotype IgA2 and/or the anti-viral vector antibody of class IgG is of isotype IgG1, isotype IgG2, isotype IgG3 or isotype IgG4.

Embodiment 6. The method of any one of embodiments 3-5, further comprising contacting the incubated mixture with a complement system protein that is capable of binding to the anti-viral vector antibody associated with the viral vector covalently immobilized on the SPR sensor chip.

Embodiment 7. The method of embodiment 6, wherein the complement system protein is C1q.

Embodiment 8. The method of embodiment 6 or 7, further comprising determining the binding level of the complement system protein to the anti-viral vector antibody associated with the viral vector, wherein the binding level is indicative of the level of activation of the complement system.

Embodiment 9. The method of any one of embodiments 1-8, wherein the viral vector is an adeno-associated viral vector.

Embodiment 10. The method of embodiment 9, wherein the adeno-associated viral vector is AAV1, AAV2, AAV4, AAV5, AAV6, AAV7, AAV8, or AAV9.

Embodiment 11. The method of embodiment 9 or 10, wherein the adeno-associated viral vector is AAV2, AAV5, AAV8, or AAV9.

Embodiment 12. The method of any one of embodiments 1-11, wherein the viral vector comprises a reporter.

Embodiment 13. The method of embodiment 12, wherein the reporter is a fluorescent, colorimetric, or chemiluminescent reporter.

Embodiment 14. The method of embodiment 12 or 13, wherein the reporter is luciferase.

Embodiment 15. The method of any one of embodiments 1-14, wherein using SPR comprises determining a rate of association of the viral vector to the ligand, determining a binding affinity of the viral vector to the ligand, determining a rate of dissociation of the viral vector and the ligand, determining a rate of regeneration of a ligand.

Embodiment 16. The method of any one of embodiments 2-15, further comprising diluting the biological sample, determining the extent to which the viral vector associates with the ligand in the diluted biological sample, and comparing the extent of such association in the diluted biological sample.

Embodiment 17. The method of embodiment 16, further comprising determining the concentration of the anti-viral vector antibody in the diluted biological sample and/or titering the anti-viral vector antibody in the diluted biological sample.

Embodiment 18. The method of any one of embodiments 1-17, wherein the biological sample is serum, plasma, whole blood, umbilical cord blood, cerebrospinal fluid, intraocular fluid, synovial fluid, saliva, bronchial fluid, alveolar fluid, gastrointestinal lavage fluids, or urine.

Embodiment 19. The method of any one of embodiments 1-18, wherein the ligand is immobilized on a solid support and the mixture is flowed over the solid support.

Embodiment 20. The method of embodiment 19, wherein the ligand is covalently attached to the solid support.

Embodiment 21. The method of embodiment 20, wherein the ligand is attached to the solid support by affinity binding.

Embodiment 22. The method of any one of embodiments 1-21, wherein the solid support comprises long and/or short chain dextran, carboxymethyl dextran, alginate, immobilized streptavidin, immobilized Protein A, or immobilized nitrilotriacetic acid (NTA).

Embodiment 23. The method of any one of embodiments 1-22, wherein the ligand is laminin receptor, heparan sulfate proteoglycans, AAV receptor or an antibody specific to the viral vector.

Embodiment 24. The method of any one of embodiments 1-23, further comprising using SPR to determine association of the viral vector with the ligand in the presence of a positive control sample known to comprise an anti-viral vector antibody, and comparing the association of the viral vector with the ligand in the presence of the biological sample to the association of the viral vector with the ligand in the presence of the positive control sample.

Embodiment 25. The method of claim 24, wherein the positive control sample comprises a purified anti-viral vector antibody of known titer.

Embodiment 26. The method of claim 25, wherein the purified anti-viral vector antibody is of known affinity for the viral vector.

Embodiment 27. The method of claim 26 or 27, wherein the purified anti-viral vector antibody is of known avidity for the viral vector.

Embodiment 28. The method of any one of embodiments 24-27, wherein comparing the association of the viral vector with the ligand in the presence of the biological sample to that in the presence of the positive control sample results in a quantitative measurement of the presence of an anti-viral vector antibody against the viral vector in the biological sample.

Embodiment 29. The method of any one of embodiments 1-28, wherein using surface plasmon resonance to determine association of the viral vector with the ligand comprises assessing inhibition of the association by the anti-viral vector antibody in the biological sample or in the positive control sample based on maximal binding of the viral vector (Rmax) values measured for a plurality of dilutions of the biological sample or the control sample.

Embodiment 30. A method comprising:
obtaining a biological sample from each of multiple subjects suspected to be in need of treatment with a viral vector-based gene therapy composition;
performing the method according to any one of embodiments 1-28 on the biological sample; and
stratifying the subjects based on the presence of the anti-viral vector antibody in the biological sample.

Embodiment 31. The method of claim 30, wherein the subjects having the biological sample determined to be negative for the anti-viral vector antibody or below a predetermined threshold for anti-viral vector antibody are selected for treatment with the viral vector-based gene therapy composition.

Embodiment 32. The method of claim 31, further comprising excluding from treatment with the viral vector-based gene therapy composition subjects having the biological sample determined to be positive for or above a predetermined threshold for the anti-viral vector antibody.

Embodiment 33. The method of claim 31 or 32, wherein the method comprises providing a companion diagnostic for use with the viral vector-based gene therapy composition.

### EXAMPLES

The following examples are provided to further describe some of the embodiments disclosed herein. The examples are intended to illustrate, not to limit, the disclosed embodiments.

Materials and Methods

Reagents and Materials

The dextran-based CM5 sensor chip, the Acetate 4.0 immobilization buffer, the Amine Immobilization Kit, the HBS-EP Running buffer, the Glycine pH 1.5 regeneration solution were all purchased from GE Healthcare Life Sciences. The custom made purified AAV8 virus-luciferase vector was obtained from Vigene Biosciences. The anti-AAV8-specific neutralizing mouse monoclonal IgG2a antibody ADK8 was purchased from Progen. The goat anti-mouse IgG2a antibody was purchased from Sigma. Human anti-AAV8 positive and negative sera were obtained from Bio-IVT. The SPR instrument used was GE's Biacore T100 with the T200 upgrade. Anti-TAb cocktail containing purified antibodies to human IgA, IgG and IgM was purchased from Novus Biologicals (Centennial, Colo.). Purified human C1q protein was purchased from Complement Technologies (Tyler, Tex.).

Human serum samples were purchased from IBT Bioservices (Rockville, Md.).

These serum samples were tested for NAb activity using a cell-based assay (Meliani et al., Human Gene Therapy Methods, 26, 45-53, 2015). Five samples highly positive for NAb activity and NAb negative serum were selected as shown in Table 1.

TABLE 1

| | NAb inhibitory activity in cell-based assay | | | | |
|---|---|---|---|---|---|
| | | | MEAN | % maximal | |
| | Norm. | Norm. | RLU | Transduction | |
| Sample ID | RLU#1 | RLU#2 | signal | Inhibition | % CV |
| BRH1454897 | 0.008 | 0.005 | 0.007 | 99.1 | 27.1% |
| BRH1454903 | 0.006 | 0.005 | 0.006 | 99.2 | 10.9% |
| BRH1454904 | 0.007 | 0.010 | 0.009 | 98.8 | 23.6% |
| BRH1454910 | 0.009 | 0.011 | 0.010 | 98.7 | 10.0% |
| BRH1454926 | 0.011 | 0.008 | 0.010 | 98.7 | 21.9% |

NAb negative control (NC Pool) >0.78 0

Immobilization of the AAV8-Luciferase Virus Vector to the Sensor Chin

The AAV8 vector was bound to the Biacore CM5 sensor chip using the Acetate 4.0 buffer. This was followed by the covalent coupling of the virus using Biacore's amine immobilization kit. This procedure created a stable virus surface that was repeatedly used (>50 cycles) after regeneration with the glycine pH 1.5 regeneration solution.

ADK8 Antibody Binding and Confirmation.

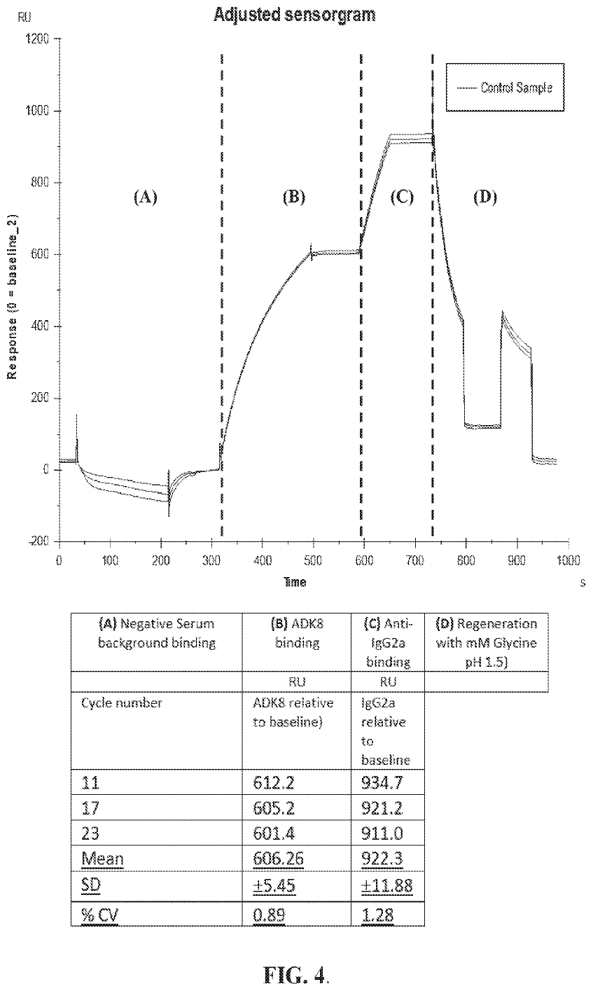

The NAb negative human serum pool was diluted 1:20 with the HBS-N running buffer. Diluted serum pool was spiked with 10 μg/mL of neutralizing anti-AAV8 (ADK8 mouse monoclonal IgG2a) and in a separate sample with 10 μg/mL of the isotype specific anti-mouse IgG2a antibody. Thirty microliter samples of diluted unspiked negative serum pool as well as ADK8-spiked and anti-IgG2a-spiked serum pool were injected sequentially and flowed across the AAV8 virus surface at 10 microliters per minute to allow for interaction of each sample with the virus surface. The sensor surface was then regenerated using two cycles of 30 microliters of glycine pH 1.5 solution at 30 microliters per minute to prepare it for the next experiment. This experiment was repeated three times (cycles 11, 17 and 23) to generate three separate sensograms (See FIG. 4).

Detection of Neutralizing Anti-AAV8 Antibodies in Human Serum.

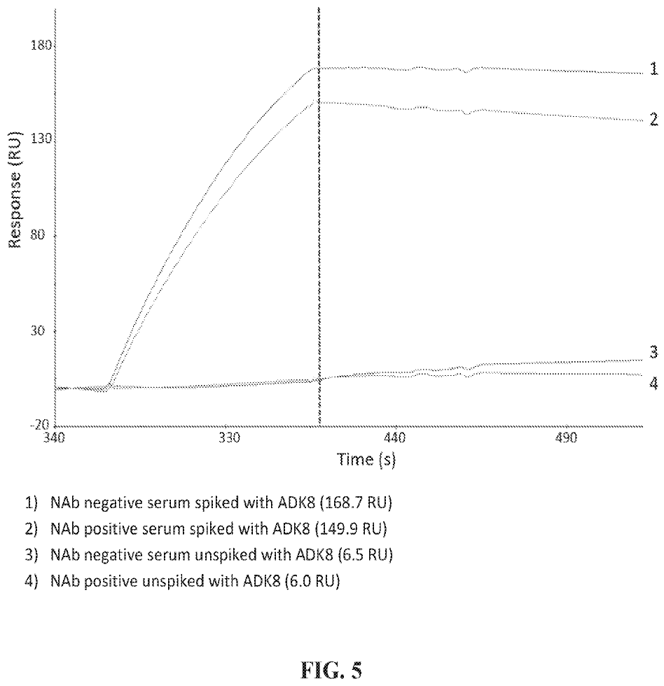

Neutralizing anti-AAV8 antibodies in human sera were detected by competitive inhibition of the ADK8 binding to the AAV8 virus surface. NAb negative and NAb positive serum samples were selected based on the HEK 293T cell transduction with the AAV8-luciferase vector (Meliani et al., Human Gene Therapy Methods, 26, 45-53, 2015). Each serum was diluted 1:20 with the HBS-N Running buffer. One NAb positive sample was spiked with 10 μg/mL of the ADK8 antibody and the other was left unspiked. The same was done with the NAb negative serum. Thirty microliter samples of ADK8 spiked and unspiked sera were injected and flowed across the AAV8 virus surface at 10 microliters per minute to allow for interaction of each sample with the virus surface. This was done with both NAb positive and NAb negative sera. Following injection of each serum sample, 30 μL of the anti-IgG2a antibody diluted in the Running buffer at concentration of 10 μg/mL was injected and flowed across the sensor chip at 10 microliters per minute to allow for detection of the bound ADK8 antibody. This experiment resulted in 4 different sensograms (1-4) shown in FIG. 5.

Binding of human anti-AAVantibodies (TAb) toAAV8 virus chin and confirmation with secondary anti-human immunoglobulins. The NAb negative human serum pool (NC pool) and five different NAb positive sera were diluted 1:20 with the HBS-N running buffer. Thirty microliter serum samples were separately flowed across the AAV8 virus surface at 30 microliters per minute for 180 seconds followed by 60 seconds of equilibration to allow for interaction of each sample with the virus surface. This was followed by 60 microliters of HBS-N running buffer and injection of 30 microliters anti-human IgA/G/M cocktail (Novus Biologicals, 10 ug/mL) at the same flow rate. After each serum plus anti-human IgA/G/M run, the AAV8 sensor surface was regenerated using two cycles of 30 microliters of 10 mM glycine pH 1.5 solution at 30 microliters per minute to prepare it for the next experiment.

Binding of C1q to human anti-AAV antibodies associated with the AAV8 virus chip. The NAb negative human serum pool (NC pool) and five different NAb positive sera were diluted 1:20 with the HBS-N running buffer. Thirty microliter serum samples were separately flowed across the AAV8 virus surface at 30 microliters per minute for 180 seconds followed by 60 seconds of equilibration to allow for interaction of each sample with the virus surface. This was followed by 60 microliters of HBS-N running buffer and injection of 30 microliters purified human C1q protein (Complement Technologies, 10 ug/mL) at the same flow rate. After each serum plus C1q run, the AAV8 sensor surface was regenerated using two cycles of 30 microliters of glycine pH 1.5 solution at 30 microliters per minute to prepare it for the next experiment.

### Example 1: Anti-Viral Vector Antibodies Detection Via SPR Assay Based on AAV-Specific Ligand

The following is an example protocol that is expected to allow for conclusive comparison of competitive SPR results with cell-based transduction inhibition methods of detecting anti-viral vector antibodies in a biological sample.

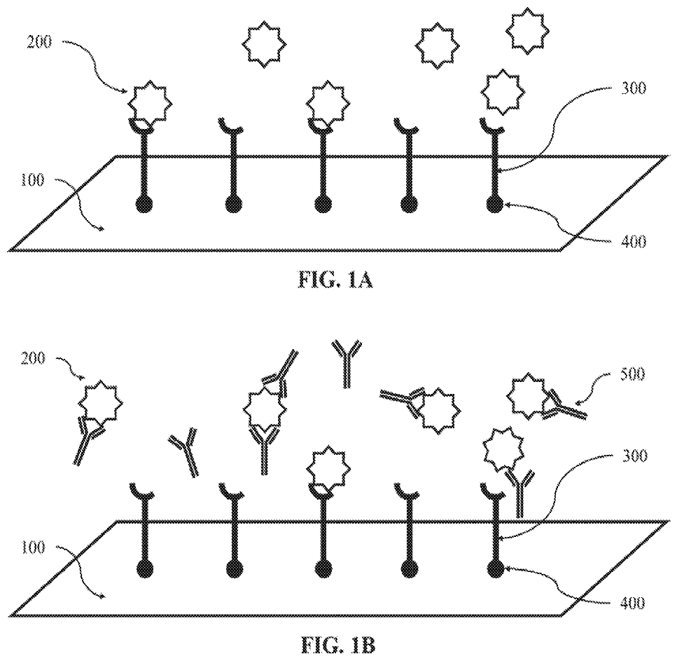

An AAV receptor or an AAV ligand is immobilized on a solid support (FIG. 1A). The solid support is a target conductance surface, which can be, for example, a Biacore CM5 chip. The ligand can be heparan sulfate proteoglycans, AAV receptor, the laminin receptor, or an antibody specific for an AAV serotype. The ligand can be covalently conjugated to the solid support. Methods of conjugating ligands to SPR-amenable solid supports include, for example: amine coupling; carbodiimide coupling; glutaryl aldehyde cross-linking; sulfhydryl cross-linking; cyanogen bromide activated coupling; chelation coupling; biotin-avidin/biotin-streptavidin binding; immunoglobulin-protein A/G/L binding; or polyhistidine-NTA binding.

An AAV ligand is contacted with or flowed over by an AAV. The AAV can be of a specific serotype. The serotype of the AAV vector can direct which ligand is used. The AAV can have a reporter, for example a luciferase reporter or a gene encoding luciferase. The AAV can be a commercially available AAV-luciferase vector. The AAV is added at different concentrations of vector particles to the immobilized receptor to establish optimal virus sensogram with highest reproducible Rmax.

Once determined, the optimal concentration of the AAV vector is preincubated with increasing concentrations of the positive control anti-viral vector antibody and contacted with or flowed over the immobilized receptor (FIG. 1B) to generate and explore competitive inhibition sensograms.

In parallel, the same AAV vector is optimized in a suitable cell-based transduction assay as described elsewhere and as known to persons having ordinary skill in the art. The optimal concentration of the AAV is preincubated with increasing concentrations of the positive control anti-viral vector antibody, and transduction inhibition effects caused by the antibody are determined according to known cell-based detection methods or methods described elsewhere.

Competitive inhibition effect of the anti-viral vector antibodies is determined in the cell-based method and in the SPR method.

To characterize anti-viral vector antibodies in unknown samples (e.g., serum samples), the optimized AAV vector is preincubated with dilutions of the unknown samples and contacted with or flowed over the immobilized ligand.

Competitive SPR sensograms (illustrated in FIG. 3) are generated to determine inhibitory effects of serum dilutions based on Rmax values measured for each serum dilution. As shown in FIG. 3, competitive sensograms are expected to show gradual increase in the maximal binding, i.e., Rmax, of the AAV vector to the immobilized ligand in response to increasing serum dilutions where the concentration of anti-viral vector antibodies (e.g. neutralizing antibodies, NAbs) is decreasing in proportion with increasing dilutions. The bottom sensogram represents the lowest dilution of the hypothetical NAb positive serum (e.g. 1:2), whereas the top sensogram represents the binding of AAV to the immobilized AAV ligand in the absence of serum.

Rmax values are plotted against corresponding serial dilutions using a linear or 4-parameter fitting equation per (or alternative) algorithm.

The “50% inhibition titer” is determined using the Rmax vs. serum dilution plot. The 50% inhibition titer represents the serum anti-viral vector antibody capacity. An alternative approach to the assessment of the serum anti-viral vector antibody capacity involves determining the minimum required dilution (MRD) of the serum to be used in the competitive SPR method. Using the established MRD, a number of samples (e.g., 100-200 normal donors) is screened with the above competitive SPR protocol to identify and select 50-60 anti-viral vector antibody-negative samples for the AAV vector or any other vector intended as a gene therapy composition.

Appropriate statistical analysis of negative samples can determine a screening cut point to be used in distinguishing positive from negative samples from the unknown population.

All anti-viral vector antibody-positive samples can be titrated to determine the end point titer as a measure of the serum anti-viral vector antibody capacity.

One can establish an immunoglobulin depletion step using commercial protein A/G/L beads to confirm the specificity of anti-viral vector antibody signal detected in unknown serum samples irrespective of which of the two methods (SPR or cell-based) is used for measurement of anti-viral vector antibody capacity.

Finally, run the luciferase cell-based transduction assay with the same AAV serotype luciferase vector as well as the same serum dilutions used in the competitive SPR and compare inhibitory effects determined in both methods. The SPR method is thereby optimized, validated, and calibrated.

### Example 2: Anti-Viral Vector Antibodies Detection Via SPR Assay Based on AAV-Specific Antibody

The following is an alternative protocol to that provided in Example 1. The protocol is expected to allow for conclusive comparison of competitive SPR results with cell-based transduction inhibition methods of detecting anti-viral vector antibodies in a biological sample.

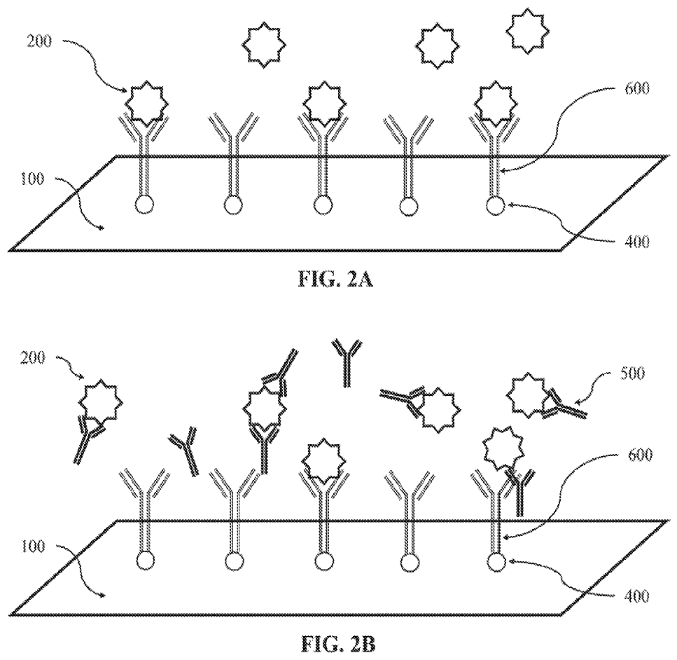

Anti-AAV positive control antibodies are immobilized on a solid support (FIG. 2A), for example on Biacore CM5 chip.

An AAV vector is contacted with or flowed over the immobilized anti-AAV positive control anti-viral vector antibody to establish optimal virus sensogram.

The optimal AAV vector concentration is preincubated with increasing concentrations of the positive control anti-viral vector antibody and contacted with or flowed over the immobilized positive control anti-viral vector antibody (FIG. 2B) to generate and explore competitive inhibition sensograms.

In parallel, a cell-based transduction assay is optimized with the same AAV vector.

The optimal AAV vector concentration is preincubated with increasing concentrations of a positive control anti-viral vector antibody. Transduction inhibition effects caused by the antibody are determined by cell-based method and the competitive inhibition of AAV vector binding by the SPR method.

For unknown serum samples, optimal concentration of the AAV vector are preincubate the with various serum dilutions and contacted with or flowed over the immobilized positive control anti-viral vector antibody.

Competitive SPR sensograms are generated to determine inhibitory effects of serum dilutions analogous to the methods described in Example 1.

The cell-based transduction assay is performed with the same AAV vector as well as the same serum dilutions used in the competitive SPR method and inhibitory effects are determined in both methods and compared.

### Example 3: Characterization of Anti-AAV Antibodies by SPR

Neutralizing anti-AAV IgG antibodies (NAbs) have been shown to interfere with AAV vector uptake by target cells and thereby inhibit gene transduction. Recent experimental data from nonhuman primates and humans demonstrated that clinically approved procedures (e.g. Therasorb) for removal of immunoglobulins from plasma can remove NAbs and, thereby, enhance AAV mediated gene transduction (Salas et al., Blood Advances, 3, 2632-2641, 2019). However, these procedures remove not only IgG but also IgA, IgM, and IgE as well as complement components. It has been also shown that IgM as well as individual complement factors have neutralizing effects on viruses such as hepatitis C, vesicular stomatitis and adenovirus type 5 (Brenner P. et al., Eur J Cardiothorac Surg. 15, 672-679, 1999; Meyer et al., J Virol. 76, 2150-2158, 2002; Beebe et al., J Immunol. 126,1562-1568, 1981 and Qiu et al., J Virol. 89, 3412-3416, 2015). Therefore, it is conceivable that a decrease of anti-AAV neutralizing activity following immunoadsorption depends not only on depletion of NAbs but also on other, non-neutralizing antibodies and complement components. Most recently, Solid Biosciences announced that a patient in its second cohort (dosed in October 2019 with the SGT 001; an AAV9-microdystrophin vector) experienced a serious adverse event that was deemed related to the drug. The serious adverse event was characterized by complement activation, a decrease in red blood cell count, acute kidney injury and cardio-pulmonary insufficiency. As a result, the FDA placed the clinical hold on the trial (Keown A. BioSpace, Nov. 12, 2019).

Disclosed herein are methods for detecting and characterizing all pre-existing and treatment induced anti-AAV antibodies. Immobilized AAV sensor chips are useful not only for neutralizing antibodies anti-AAV (NAbs) but also for detection and characterization total binding anti-AAV antibodies (TAbs) in human serum.

Main Steps for Determining the Presence and Titer of Anti-AAV Immunoglobulin Classes and Isotypes Thereof:
1) Immobilize selected AAV serotype (AAV1-AAV9) by covalent coupling to biosensor chip of choice (e.g. CM5);
2) Flow serum sample over the immobilized virus
3) Monitor sensogram until a stable sample response is achieved
4) For antibody classes, sequentially flow anti-human antibodies specific for IgA, IgE, IgG and IgM
5) For antibody isotypes sequentially flow anti-human antibodies specific for IgG1, IgG2, IgG3, IgG4, IgD, IgA1 and IgA 2, over the bound serum anti-AAV antibodies without regenerating the chip surface in between antibodies
6) Monitor sensogram until stable confirmatory response is achieved after each class or isotype specific anti-human antibody
7) When the SPR signal increases for a specific anti-class or anti-isotype antibody, it is confirmed that anti-AAV antibody in a given serum sample belongs to the corresponding class or isotype. Results obtained with the mouse anti-AAV8 monoclonal IgG2a antibody (ADK8) confirmed with the anti-mouse IgG2a isotype specific antibody demonstrate feasibility of isotyping by the SPR (FIG. 4)
8) Regenerate immobilized AAV surface
9) Repeat steps 2-7 using different serum dilutions to determine the end point titer for each class or isotype defined as the highest dilution that results with a confirmatory response measurable above the background

### Affinity and Avidity of Anti-AAV TAbs and Isotypes:

Affinity and avidity of anti-AAV antibodies are important characteristics thereof because they influence binding efficiency (affinity) and stability (avidity) of antibody-virus complexes. Anti-AAV antibodies of higher avidity are generally more likely to interfere with transduction because of their sustained presence on the vector surface. Low avidity antibodies that readily dissociate from the virus may not reside on the vector surface long enough to affect transduction. It is, therefore, important to determine affinity and avidity each anti-AAV antibody class and isotype. Binding kinetics of the serum anti-AAV antibody will be analyzed using the corresponding portion of the SPR sensogram. Association rate constant (ka, also termed Kon), dissociation rate constant (kd, also termed Koff) as well as affinity constant Kd will be determined. This can be accomplished using published Biacore™ algorithms and protocols (GE Healthcare Lifesciences, Biacore™ Assay Handbook, Appendix A: Analysis of kinetics and concentration measurements; and van der Merwe, Surface plasmon resonance, Biophysics. Biochem. Cambridge Univ., 2011).

As shown in FIG. 4, anti-AAV8-specific neutralizing mouse monoclonal IgG2a antibody (ADK8) binds to AAV8 virus sensor chip and its association with the virus is confirmed with anti-mouse isotype specific antibody against IgG2a. All three sensograms displayed four different events (A, B, C, D) in a practically overlapping pattern. Section A showed virtually no binding of the unspiked negative serum pool to the virus surface representing the sensor chip background. Section B showed significant binding of ADK8 relative to baseline with the mean RU of 606.26±5.45 SD and 0.89% CV. Section C showed binding of anti-mouse IgG2a confirmatory antibody with the mean RU of 922.3±11.88 SD and 1.28% CV relative to baseline, which included additive ADK8 and confirmatory anti-IgG2a binding. Section D demonstrated regeneration of the virus sensor chip. Thus, the three overlapping sensograms resulting from three separate experiments and corresponding RU values thereof (FIG. 4 sections B and C) demonstrated highly reproducible ADK8 NAb binding as well as the confirmatory anti-IgG2a binding to the AAV8 virus sensor chip. Note the very low SDs and percentage CVs. The range between the highest and lowest RU values was 2% for the ADK8 and 3% for the confirmatory anti-IgG2a binding.

As shown in FIG. 5, an anti-Mouse IgG2a response is detected in serum samples spiked with ADK8. Four sensograms were generated: (1) NAb negative serum spiked with ADK8 (168.7 RU); (2) NAb positive serum spiked with ADK8 (149.9 RU); (3) NAb negative serum unspiked with ADK8 (6.5 RU) and (4) NAb positive unspiked with ADK8 (6.0 RU). Comparison of the four sensograms and corresponding RU values showed that:
1) Highest binding of the anti-mouse IgG2a antibody was found in the NAb negative serum spiked with ADK8 (sensogram 1, 168.7 RU) demonstrating the highest detectable level of ADK8 in absence of the endogenous serum NAb.
2) Binding of the anti-mouse IgG2a in the NAb positive serum spiked with ADK8 (sensogram 2, 149.9 RU) was significantly lower exceeding the method variability (FIG. 4, section B) by fourfold. In this sample, the endogenous serum NAb (proven by the cell-based assay) competed with and inhibited binding of ADK8 to the immobilized virus.
3) Both NAb positive and NAb negative sera unspiked with ADK8 (sensograms 3 and 4, RUs 6.5 and 6.0 respectively) showed only the sensor chip background without measurable anti-mouse IgG2a binding.

In summary, the results illustrated in FIGS. 4 and 5 demonstrated that:
1) AAV8 virus can be covalently immobilized on dextran coated Biacore CM5 SPR chip to provide a functional target for anti-AAV antibodies.
2) The immobilized virus surface was stable and can be regenerated with glycine pH 1.5 solution and repeatedly used (>50 regeneration cycles).
3) Anti-AAV8 NAb ADK8 (mouse IgG2a) can bind to the immobilized AAV8 virus surface.
4) Binding of ADK8 can be confirmed by sequential injection of the isotype specific (anti-mouse IgG2a) secondary antibody.
5) ADK8 NAb can bind to the immobilized virus in absence or presence of human serum.
6) Endogenous NAb in human serum inhibited the binding of ADK8 by competition for the immobilized AAV8 virus.
7) SPR NAb analysis was semi-automated and was completed in approximately 6 hours which is much shorter time than the usual 45 hours required for the cell-based assay.
8) SPR analysis was more sensitive than cell-based assays because it detected anti-AAV antibody at the 1:20 dilution of serum while cell-based assay usually requires serum dilution of 1:2 or 1:4.

It should be pointed out, that in the above experiments the AAV8 virus served as a ligand and ADK8 NAb and endogenous serum NAbs served as analytes. In examples 1 and 2, AAV receptors or antibodies are proposed as ligands and virus as an analyte. These two alternative approaches are not contradictory because both allow analysis of interaction between viral vectors and anti-vector antibodies by SPR. In fact, they provide evidence for SPR versatility.

Detection of NAbs against other AAV serotypes such as AAV5 and AAV9 by the competitive SPR described herein should be possible as corresponding NAbs are available. Furthermore, the present data suggested that immobilized AAV sensor chips can be used not only for Nabs, but also for detection and characterization TAb in human serum. By analogy to binding of ADK8 to the immobilized AAV8 and its confirmation with the anti-IgG2a (shown in FIG. 4), binding and confirmation of any other anti-AAV antibody to any AAV sensor should also be feasible. Furthermore, using an immobilized virus sensor chip would allow for comprehensive characterization of anti AAV antibodies in terms of class and isotype, affinity/avidity, titer as well as complement activating potential,

### Example 4: Characterization of Anti-AAV Mediated Complement Activation

Many anti-viral antibodies are known to activate complement upon their binding to virus particles. This activation can lead to virolysis depending on antibody titers and/or isotypes (Oroszlan et al., Science, 168,1478-1480, 1970; Spear et al., Journal of Virology, 67, 53-59, 1993; Sharp et al., PNAS, 116, 11900-11905, 2019; and Kishore et al., Immunopharmacology, 49, 159-170, 2000). The first component of complement (C1) is composed of 3 subunits designated as C1q, C1r, and C1s. C1q recognizes and binds to immunoglobulin complexed with an antigen and initiates the classical complement pathway (Kishore et al., Immunopharmacology, 49, 159-170, 2000). From the AAV virus (vector) prospective it is important to determine the complement activating potential of anti-AAV antibodies in order to assess the risk of diminished transduction as well as overall safety risks associated with complement activation. To determine the complement activating potential of pre-existing and treatment induced anti-AAV antibodies, binding of the C1q complement to the antibody-virus complexes will be analyzed. The magnitude and kinetics (kon, koff, Kd) of the C1q binding to antibody-virus complexes will determine the complement activating potential of a given anti-AAV antibody.

Main steps for determining the binding level of the C1q to anti-AA V antibodies bound to AAV surface:
1) Immobilize selected AAV serotype (AAV1-AAV9) by covalent coupling to biosensor chip of choice (e.g. CM5)
2) Flow a serum sample over the immobilized virus
3) Monitor sensogram until a stable serum sample response is achieved
4) Confirm the total antibody bound using anti-human IgA/G/M cocktail
5) Determine immunoglobulin class and/or isotype of the antibody bound to the AAV surface per steps 4-7 described in Determination of immunoglobulin classes and isotypes section
6) Regenerate the AAV surface
7) Re-flow the serum sample with demonstrated total antibody anti-AAV antibody presence or known antibody class/isotype over the AAV surface and monitor the sensogram until a stable serum sample response is achieved
8) Flow the native human C1q over the bound anti-AAV antibody without in between surface regeneration
9) Monitor the sensogram until a stable C1q response is achieved
10) Using the C1q portion of the sensogram, determine the magnitude and kinetics of the C1q binding

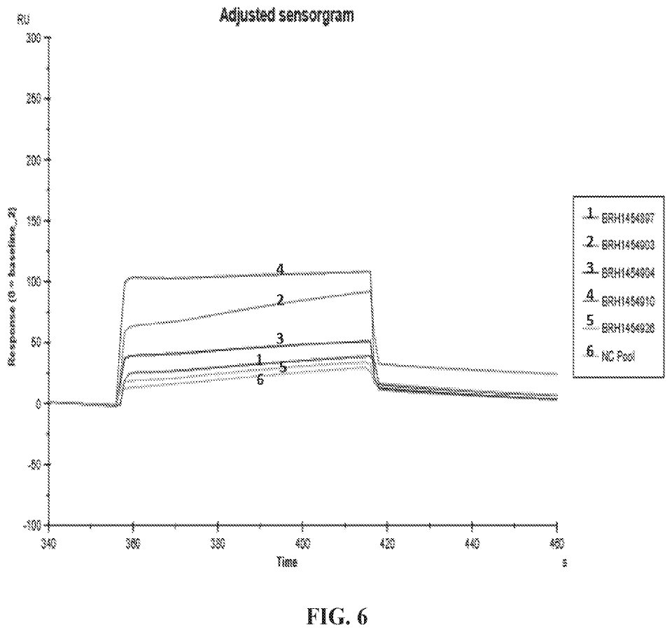

As shown in FIG. 6, anti-human IgA/G/M cocktail reacted with human serum antibodies bound to the immobilized AAV8 virus chip. These results demonstrated that detection of different levels of human antibodies bound to the immobilized whole AAV virus is feasible by using the disclosed SPR method. While the NAb inhibitory activity in all positive samples and serum samples was uniformly high (98.7 to 99.1% transduction inhibition, Table 1), the reactivity with the anti-IgA/G/M cocktail was spread over a wider range in all samples from approximately 28 to 107 response units (RUs) including the NC Pool. Furthermore, there was no perfect match between NAb activity and IgA/G/M reactivity. These results demonstrated the complexity of anti-AAV8 antibodies in tested samples and indicated the presence of antibodies other than NAbs.

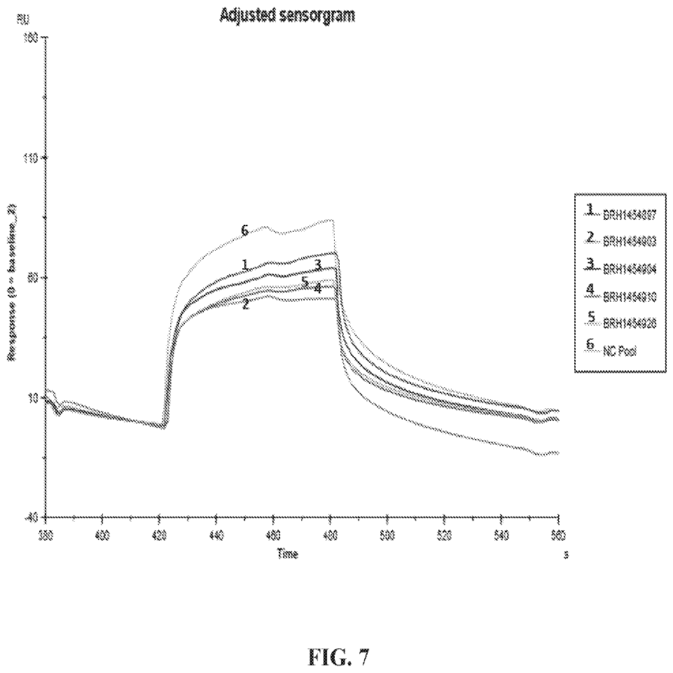

As shown in FIG. 7, human C1q interacted with human serum antibodies bound to the immobilized AAV8 virus chip. There was no match between C1q reactivity, IgA/G/M response and NAb inhibitory activity. C1q data indicated the complexity of anti-AAV8 antibodies and complement activating potential thereof. The NC pool was of particular interest as it was selected based on its lack of neutralizing activity in the cell-based assay but showed low level of reactivity with the IgA/G/M cocktail and the strongest reactivity with C1q. The most likely explanation for this observation is that the NC Pool contained low level of IgM without neutralizing activity but provided strong reactivity with C1q, which is generally known for IgM. Greater amount of C1q complement on the virus surface is consistent with stronger complement activation and, consequently, virolysis and/or virus clearance which can jeopardize gene delivery by viral vectors in combination with NAbs or independently.

In conclusion, the present results demonstrated that the methods of using SPR as disclosed allow:
1) The detection and measurement of human antibodies bound to AAV viruses immobilized on Biacore chip.
2) The analysis and characterization of anti-AAV antibodies in human serum in terms of immunoglobulin classes and isotypes
3) The determination of complement activating potential of anti-AAV antibodies via C1q binding to antibodies bound to the immobilized virus
4) The analysis of complex mixtures of neutralizing and non-neutralizing antibodies present at various levels and with variable capacity to bind C1q and potential for complement activation

The disclosures of each and every patent, patent application, and publication cited herein are hereby incorporated herein by reference in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

We claim:

1. A method for detecting an anti-viral vector antibody, the method comprising:
incubating a mixture that comprises a viral vector and a biological sample suspected of containing an anti-viral vector antibody against the viral vector;
contacting the mixture with a ligand for the viral vector; and
using surface plasmon resonance (SPR) sensor chip to determine the extent to which the viral vector associates with the ligand following said contacting step;
wherein association of the viral vector with the ligand is reduced in the presence of the anti-viral vector antibody.

2. The method of claim 1, further comprising using SPR to determine association of the viral vector with the ligand in the presence of a negative control sample known to lack an anti-viral vector antibody against the viral vector, and comparing the association of the viral vector with the ligand in the presence of the biological sample to that in the presence of the negative control sample.

3. The method of claim 1, wherein the anti-viral vector antibody is a neutralizing antibody.

4. The method of claim 1, wherein the anti-viral vector antibody is an immunoglobin (Ig) of class A (IgA), class D (IgD), class E (IgE), class G (IgG) or class M (IgM).

5. (canceled)

6. The method of claim 3, further comprising contacting the mixture with a complement system protein that is capable of binding to the anti-viral vector antibody associated with the viral vector covalently immobilized on the SPR sensor chip, wherein the complement system protein is C1q.

7. (canceled)

8. The method of claim 6, further comprising determining binding level of the complement system protein to the anti-viral vector antibody associated with the viral vector, wherein the binding level is indicative of the level of activation of the complement system.

9. The method of claim 1, wherein the viral vector is an adeno-associated viral vector selected from the group consisting of AAV1, AAV2, AAV4, AAV5, AAV6, AAV7, AAV8, and AAV9.

10. (canceled)

11. (canceled)

12. The method of claim 1, wherein the viral vector comprises a reporter selected from the group consisting of a fluorescent, a colorimetric, and a chemiluminescent reporter.

13. (canceled)

14. The method of claim 12, wherein the reporter is luciferase.

15. The method of claim 1, wherein using SPR comprises determining a rate of association of the viral vector to the ligand, determining a binding affinity of the viral vector to the ligand, determining a rate of dissociation of the viral vector and the ligand, and/or determining a rate of regeneration of a ligand.

16. (canceled)

17. (canceled)

18. The method of claim 1, wherein the biological sample is serum, plasma, whole blood, umbilical cord blood, cerebrospinal fluid, intraocular fluid, synovial fluid, saliva, bronchial fluid, alveolar fluid, gastrointestinal lavage fluids, or urine, optionally, wherein the biological sample is a diluted biological sample.

19. The method of claim 1, wherein the ligand is immobilized on a solid support covalently or by affinity binding and the mixture is flowed over the solid support.

20. (canceled)

21. (canceled)

22. The method of claim 19, wherein the solid support comprises long and/or short chain dextran, carboxymethyl dextran, alginate, immobilized streptavidin, immobilized Protein A, or immobilized nitrilotriacetic acid (NTA).

23. The method of claim 1, wherein the ligand is laminin receptor, heparan sulfate proteoglycans, AAV receptor or an antibody specific to the viral vector.

24. The method of claim 1, further comprising using SPR to determine association of the viral vector with the ligand in the presence of a positive control sample known to comprise an anti-viral vector antibody, and comparing the association of the viral vector with the ligand in the presence of the biological sample to the association of the viral vector with the ligand in the presence of the positive control sample, optionally, wherein the positive control sample comprises a purified anti-viral vector antibody of known titer, of known affinity for the viral vector, or of known avidity for the viral vector.

25. (canceled)

26. (canceled)

27. (canceled)

28. The method of claim 24, wherein comparing the association of the viral vector with the ligand in the presence of the biological sample to that in the presence of the positive control sample results in a quantitative measurement of the presence of an anti-viral vector antibody against the viral vector in the biological sample.

29. The method of claim 1, wherein using surface plasmon resonance to determine association of the viral vector with the ligand comprises assessing inhibition of the association by the anti-viral vector antibody in the biological sample or in the positive control sample based on maximal binding of the viral vector (Rmax) values measured for a plurality of dilutions of the biological sample or the control sample.

30. A method comprising:
obtaining a biological sample from each of multiple subjects suspected to be in need of treatment with a viral vector-based gene therapy composition;
performing the method according to claim 1 on the biological sample; and
stratifying the subjects based on the presence of the anti-viral vector antibody in the biological sample.

31. The method of claim 30, wherein the subjects having the biological sample determined to be negative for the anti-viral vector antibody or below a predetermined threshold for anti-viral vector antibody are selected for treatment with the viral vector-based gene therapy composition, and, optionally, excluding from treatment with the viral vector-based gene therapy composition subjects having the biological sample determined to be positive for or above a predetermined threshold for the anti-viral vector antibody.

32. (canceled)

33. The method of claim 31, wherein the method comprises providing a companion diagnostic for use with the viral vector-based gene therapy composition.
